(19) United States
(12) Patent Application Publication
(10) Pub. No.: US 2006/0078497 A1
(43) Pub. Date: Apr. 13, 2006

(54) Health management and monitoring with and without weight loss

(76) Inventor: James B. Johnson, Metairie, LA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/246,780

(22) Filed: Oct. 7, 2005

Related U.S. Application Data
(63) Continuation-in-part of application No. 10/844,535, filed on May 13, 2004.
(60) Provisional application No. 60/620,004, filed on Oct. 19, 2004.
(60) Provisional application No. 60/691,791, filed on Jun. 17, 2005.

(51) Int. Cl. G06Q 10/00; C12Q 1/68; A61K 49/00

(52) U.S. Cl. 424/9.1; 435/6; 705/2

(57) ABSTRACT

Health management and monitoring predicated on an intermittent calorie restricted health management paradigm is provided. In an illustrative implementation, an intermittent calorie restricted health management program is provided to a participating health management subject so improve the health state of the participating subject (e.g., impact disease states in the health management subject) with or without the participating subject observing weight loss. In the context of health monitoring, in an illustrative operation, the levels of the SIRT-1 protein are measured on the participating health management subject. The measured SIRT-1 protein levels provide an indication of whether the participating health management subject is adhering to the provided intermittent calorie restricted health management paradigm.

### PRIORITY AND CROSS REFERENCE

This application claims the benefit of and priority to, U.S. Provisional Application 60/620,004, filed on Oct. 19, 2004, entitled, “USE OF DIETARY PROCESS FOR TREATMENT OF DISEASE WITH AND WITHOUT WEIGHT LOSS,” U.S. Provisional Application 60/691,791 filed on Jun. 17, 2005, entitled, “SIRT-1 MONITORING AS PART OF HEALTH MANAGEMENT” and is a continuation-in-part of U.S. patent application Ser. No. 10/844,535, filed on May 14, 2004, entitled, “PROCESS FOR WEIGHT CONTROL AND LONGEVITY EXTENSION THROUGH DIETARY MANAGEMENT,” which are herein incorporated by reference in their entirety.

### FIELD OF THE INVENTION

The invention relates to health management and monitoring, and more particularly, to health management and monitoring as with and without weight loss that impacts disease and that employs temporal based caloric restrictions.

### BACKGROUND OF THE INVENTION

The need to control food intake among Americans has become more critical year after year. Estimates are that the average weight for adults has increased 16 pounds or more in the past twenty years. The incidence of non insulin dependent diabetes mellitus has increased rapidly, such that projections are that 29% of children will develop non-insulin dependent diabetes mellitus (NIDDM) within 15 years.

It is conjectured that the success of any weight reduction diet is dependent on compliance. The factors which affect compliance include, but are not limited to, degree of hunger sensed and psychological factors related to the sense of despair, failure, self deprecation, stress of the diet itself, the type of food consumed, and taste. The psychological mechanisms of denial and rationalization are theorized to play a major role in failure to maintain adherence to the diet. Popular diets require considerable preparation and forethought on a daily or more frequent basis. This can be daunting for many, and especially over a long period of dieting. As with exercise, there is a presumption that the enjoyment of the process greatly affects willingness to stick with a particular diet. In addition, dieters often encounter a lack of energy and face a constant fear that he/she will be exposed to temptation, which only adds to a dieter's stress.

Many studies have been performed on animals since the 1930's demonstrating a consistent increase in lifespan by the use of calorie restriction, (CR). A wide range of species has been tested, including protozoa, insects, rodents, monkeys and others. A consistent pattern of effect has been demonstrated that, as a generalization, a 40% reduction in calories leads to a 40% increase in both the average and maximum lifespan for the species. The mechanism is fully not known, but it is theorized that it relates to one or more adaptations occurring early in the evolutionary process for a broad range of species. With CR, the tested animals show consistent weight loss and lower metabolic function over time. It is accepted that the Sir2 gene in animals (known as SIRT-1 gene in humans) is activated by CR.

Data from such studies suggests that CR may contribute to extending both the average and maximum species' lifespan. Beneficial changes in physiology measured by biochemical markers are seen in CR animals. The humoral factors elaborated by the CR animal may account for at least some of the effects seen, which may be the result of the activation of possibly many different “longevity” genes. Among some beneficial effects seen from CR studies in animals are reduction in atherosclerosis, lower incidence of NIDDM, affecting conditions such as Alzheimer's and multiple sclerosis, protection against renal disease, a lower incidence of cancer and protection of the nervous system from disease and injury. Chronic inflammatory processes are also diminished. The data further suggests that humans may also respond to CR like other species. Additionally, it is known that beneficial changes occur in serum glucose and insulin levels in response to CR in animals, and data shows that alternate day fasting produced the same or greater lowering of insulin and glucose than daily 60% fed mice.

Current health management and monitoring practices allow for the modification of diet to realize desired health benefits. For example, a low fat diet can be proscribed for subjects trying to loose weight and reduce cholesterol. Although effective, such exemplary low fat diet can be effective to realize those specific desired health benefits, such diet is difficult to maintain as the participating subject is restricted in eating as they normally would. Additionally, the collateral health benefits of such exemplary diet are not fully known and it is unclear whether such exemplary diet contributes to the treatment of diseases such as cancer, Alzheimer's, and multiple sclerosis.

Additionally, current health management monitoring is generally focused on a single quantitative measure of whether a participating subject is losing or maintaining weight. In some instances, health practitioners can monitor certain biochemical levels of the subject such as cholesterol and blood sugar as part of a particular health management paradigm or protocol. The monitoring of such levels can assist health practitioners to monitor the impact of as particular health management's paradigm and/or protocol. For certain low-carbohydrate health management paradigms and protocols, a subject's ketones can be monitored through the use of ketone test strips to indicate to the subject (and/or health practitioner) whether the subject's body has entered into a ketonic state which some researchers believe is a body state that promotes weight loss. However, current practices do not provide an effective biochemical quantitative measure to indicate whether health management participants are following a calorie restricted health management paradigm or protocol.

From the foregoing it is appreciated that there exists a need for health management and monitoring systems and methods that overcome the limitations of existing practices.

### SUMMARY

A system and method are provided for health management and monitoring with and without weight loss. In an illustrative implementation, health management and the treatment of disease through health management can be realized through an application of a selected intermittent calorie restricted paradigm. The selected intermittent calorie restricted paradigm can have the effect of reducing the incidence of various diseases in human subjects. In the illustrative implementation, the intermittent calorie restricted paradigm can comprise providing a selected percentage of a total caloric intake that is consumed over a first time period T1 and providing a second percentage of the total caloric intake that is consumed over a second time period T2. The illustrative implementation further provides that time period T2 is selected to be greater than time period T1. Furthermore, in the exemplary implementation, the average of the caloric intake over time periods T1 and T2 falls within a selected percentage range of a total caloric intake.

In an illustrative operation, a participating subject can repeatedly intake the first and second selected percentages of the total caloric intake during the selected time periods T1 and T2 for a period of time T3 or until certain health management goals have been achieved. Additionally, the illustrative implementation further provides that the participating subject is able to chronicle his/her caloric intake in a journal or other memorializing media during time period T3 or until certain health management goals have been achieved. In addition, improvement in significant symptoms of a variety of diseases can be expected based on animal studies and may serve as a guide to modify the intermittent calorie restriction paradigm as needed.

Further, in an illustrative implementation, health monitoring can be realized using a quantitative measure of a bio-chemical that can result from an application of a selected intermittent calorie restricted paradigm. In the illustrative implementation, the intermittent calorie restricted paradigm can have the result of the production of SIRT-1 enzymes a participating subject. Further, in the illustrative implementation, the production of the SIRT-1 enzyme and/or a precursor to the production of the SIRT-1 can be measured through one or more tests as an indicator that a participating health management subject is adhering to the intermittent calorie restricted paradigm.

In an illustrative operation, a participating health management subject can be subjected to an intermittent calorie restricted paradigm. In the illustrative operation, the participating health management subject is monitored through one or more tests (e.g., biochemical test) to ascertain the level of the SIRT-1 enzyme and/or a precursor to the production of SIRT-1 in the participating health management subject. Depending on the results of the test (e.g., if SIRT-1/precursor levels reach a selected threshold), health management counselors can add, change, or delete one or more components of intermittent calorie restricted paradigm to meet selected health management goals.

Additionally, in the illustrative operation, a participating health management subject can perform one or more tests to determine the levels of the SIRT-1 enzyme and/or a precursor to the production of SIRT-1 to determine if they are adhering according to the parameters of the exemplary intermittent calorie restricted paradigm. Moreover, SIRT-1/precursor levels can be monitored as part of an exemplary process to treat disease. In an illustrative implementation, the SIRT-1/precursor levels of participating subject can be monitored as an indication of whether one or more diseases are being treated and are placed in submission. In this illustrative implementation, a correlation can be made that with increasing levels of SIRT-1/precursor disease states be reduced.

Other features and aspects of the herein described systems and methods are further described below.

### BRIEF DESCRIPTION OF THE DRAWINGS

For the purpose of illustrating the invention, there is shown in the drawings illustrative implementations of the herein described systems and methods. It is appreciated that the herein described systems and methods are not limited to the precise arrangements and instrumentalities shown. The drawings are not necessarily to scale, emphasis instead being placed on illustrating the principles of the herein described systems and methods in which:

FIG. 1 is a block diagram showing a current illustrative health management approach;

FIG. 2 is a block diagram showing another current illustrative health management approach;

FIG. 3 is a block diagram of an illustrative health management system and the cooperation of its components in accordance with the herein described systems and methods;

FIG. 4 is a block diagram showing an illustrative implementation of a health management approach based on an intermittent calorie restricted paradigm in accordance with the herein described systems and methods;

FIG. 5 is a block diagram showing another illustrative implementation of a health management approach based on an intermittent calorie restricted paradigm in accordance with the herein described systems and methods;

FIG. 6 is a flowchart showing illustrative health management processing that is performed in accordance with the herein described illustrative implementations;

FIG. 7 is a flowchart showing another illustrative health management processing that is performed in accordance with another herein described illustrative implementations; and

FIG. 8 is a flowchart showing illustrative health monitoring processing that is performed in accordance with the herein described illustrative implementations.

### DETAILED DESCRIPTION OF THE ILLUSTRATIVE IMPLEMENTATION

Calorie Restricted Paradigm:

Clinical data suggests that within a selected first period (e.g., 36 hours) of selected caloric restriction one or more biological mechanisms that can contribute to beneficial health results in participating subjects can be stimulated. Moreover, clinical data demonstrates that such benefits can be realized even if such stimulation is interrupted by a selected second period (e.g., 12 hours) of 100% full caloric intake. With a selected intermittent calorie restricted paradigm, physiologic effects can be set into motion during the first selected period of low consumption which can produce health benefits and increased longevity.

A wide range of disease conditions can be improved by adhering to a selected intermittent calorie restricted (CR) paradigm. Clinical observations indicate that such effects can occur in as little as 2 weeks, and continuing improvement has been observed for up to eight months after starting the selected calorie restricted paradigm. Exemplary diseases that are impacted by the selected intermittent CR paradigm include, but are not limited to, asthma, rheumatoid arthritis, various infectious processes including bacterial, fungal, and viral infections, Alzheimer's, multiple sclerosis, insulin resistance and non insulin dependent diabetes mellitus, neurological conditions associated with infectious and inflammatory conditions, IgE mediated seasonal allergies, auto-immune disease (sarcoidosis), hot flashes, calculus formations on the teeth, and chronic sinusitis as well as familial hyperlipidemias. Clinical data also suggests that participating subjects show improvement in neurological symptoms resulting from inflammatory and infectious processes which also indicates that the application of a selected intermittent CR paradigm could successfully neurological conditions (e.g., Alzheimer's and multiple sclerosis). Additionally, weight loss in participating subjects can also result from the application of a selected intermittent CR paradigm as part of a selected health management protocol.

It is conjectured and evidence has been noted in the article, “Small molecule activators of siruins extend Saccharomyces cerevisiae lifespan,” Howitz, Konrad T., et al., Nature 425, 191-196 (11 Sep. 2003); Nature AOP, published online 24 Aug. 2003, which is herein incorporated by reference in its entirety, that caloric restriction activates the enzyme Sir2 in Saccharomyces cerevisiae (brewer's yeast) and such activation can contribute to improved health states in participating subjects. Similar tests have shown that the activation of the equivalent human enzyme, SIRT-1, in cell culture enhances cell survival in the face of stresses such as ionizing radiation. It is further conjectured that SIRT-1 activation gives injured cells extra time to repair themselves and survive longer.

Clinical studies have also shown that SIRT-1, a p53 deacetylase, is strongly inhibited by the vitamin B₃ precursor nicotinamide. Such studies have shown that the increased expression of PNC1 (pyrazinamidase/nicotinamidase 1, which has been shown to encode an enzyme that deaminates nicotinamide, can be both sufficient for longevity and health management. PNC1 has been shown to activate the production of SIR2 (the animal equivalent of SIRT-1) and as such is conjectured to influence the production of SIRT-1. The relationship between nicotinamide and PNC1 is described in more detail in the article, Nature. 2003 May 8; 423(6936):125, Anderson, Rozalyn M. et al., which is herein incorporated by reference in its entirety.

As compared with intermittent calorie restrictive based health management protocols, commonly practiced health management approaches can impose significant food-type restrictions on their participating subjects and require the intake of prescription medication by participating subjects. For example, in some approaches, participating subjects are relegated to eating protein rich foods foregoing foods having significant carbohydrates. Other approaches require that their subjects forgo all protein rich foods in favor of vegetables and fruits. Yet another approach requires their subjects to intake foods on a balanced basis (e.g. 6-12 servings of carbohydrates per day, 2-4 servings of protein per day, etc.).

Also, current practices also do not provide a quantitative biochemical measure that can be used to determine if a participating subject is adhering to an intermittent calorie restricted paradigm and/or is receiving the benefits of such intermittent calorie restricted paradigm. Although certain health management paradigms (e.g., Atkins Diet) provide for a recommended measure through the monitoring of ketone levels, there is not currently a health management biochemical monitoring tool for intermittent calorie restricted paradigms.

The herein described systems and methods ameliorate the shortcomings of existing approaches by offering a health management and monitoring systems and methods for use with intermittent calorie restricted paradigms (i.e., intermittent calorie restricted health management paradigms). In an illustrative implementation an intermittent calorie restricted health management paradigm can require participating subjects to reduce caloric intake in a given time period T1 (e.g., during a first day) such that the reduced caloric intake is selected within a range from 0 to 70% of a selected total caloric intake amount (e.g., 2000 calorie/day total caloric intake). The illustrative implementation can further require that the participating subject intake a second selected percentage of the selected caloric intake amount during a second selected time period T2 (e.g., during a second day). The reduced caloric intake during T1 over the T1, T2 time period allows the participating subject to receive the benefits of controlling and reducing possible disease states.

In the illustrative health management implementation, if the participating subject ingests a percentage amount caloric intake during selected time period T2 that when averaged with the percentage amount caloric intake ingested during selected time period T1 is less than 100%, (e.g., 70% during T1 and 100% during T2—averages to 85% over T1, T2) then the participating subject can also observe weight loss in addition to health management benefits. However, if the participating subject ingests a percentage amount caloric intake during selected time period T2 that when averaged with the percentage amount caloric intake during selected time period T2 is equal to 100% (e.g., 70% during T1 and 130% during T2—averages to 100% over T1, T2) the participating subject can observe no weight loss in addition to health management benefits. In a preferred illustration, T1 can comprise a time period of 30-36 hours during which 10-70% of daily required calories (e.g., 2000 calories/day) are consumed followed by a T2 period of 12-18 hours in which 130-190% of daily required calories are consumed such that the total over T1 and T2 (e.g., 2 days or 48 hours) equals 200% of daily required calories.

In an illustrative implementation for health monitoring, selected blood based assays used to measure change in the amount or activity of a gene called SIRT-1 and/or a precursor to the production of SIRT-1 (e.g., PNC1) can be used to monitor compliance to a selected calorie restricted health management paradigms. Additionally, in the illustrative implementation the exemplary blood assays can be used to measure the therapeutic effect of the selected intermittent CR paradigm on disease. Current research indicates that SIRT-1 activation is hypothesized to be the first step in the largely unknown mechanism by which intermittent calorie restriction confers increased lifespan and delays, prevents, or improves a wide variety of diseases. Change in SIRT-1 and/or a precursor to the production of SIRT-1 activity or amount can be measured in the white blood cell nuclear protein. In the illustrative implementation, it is observed that a selected increase in the activity or amount of the SIRT-1 protein (e.g., 30% increase within four weeks from the start of the selected calorie restricted health management paradigm) and/or in the activity or amount (e.g., 30% increase within four weeks from the start of the selected calorie restricted health management paradigm) of a precursor to the production of the SIRT-1 protein (e.g., PNC1 gene) indicates compliance with a selected intermittent calorie restricted health management paradigm. It is appreciated that the term activity could mean activity in the production of the SIRT-1 protein and/or its precursors (e.g., PNC1 gene). In an illustrative operation, white blood cells can isolated from a blood sample drawn prior to starting the diet and at appropriate intervals while following the diet for use in measure in the SIRT-1 protein and/or a precursor to the production of SIRT-1.

In the illustrative implementation, the techniques used to measure increased SIRT-1/precursor activity or amount in DNA can result from various sources. For example, a SIRT-1 can be measure using nuclear materials that are monitored in a subject's organs. This method can be premised on the action on Bax to interrupt apoptosis when SIRT-1 is activated. Moreover, in the illustrative implementation, extensive animal studies show that diseases which are common to both other mammals and humans have responded to the effect of intermittent calorie restriction by preventing such diseases or delaying their onset or improving the severity of the disease. Specifically, in humans, it has been observed that the application of a selected intermittent calorie restricted health management paradigm can improve health issues including asthma, autoimmune disorders (rheumatoid arthritis), osteoarthritis, insulin resistance, inflammatory neurological disorders, hot flashes, calculus formation on the teeth, chronic sinusitis, seasonal allergies, and infectious diseases of viral, bacterial and fungal origin. Based on animal research, causes of disease in animals, for which there are corresponding diseases in humans, are amenable to treatment using a selected calorie restricted health management paradigm. As such SIRT-1 testing can be implemented as an exemplary means of monitoring the effectiveness of the selected intermittent calorie restricted health management paradigm.

Previous Health Management Approaches:

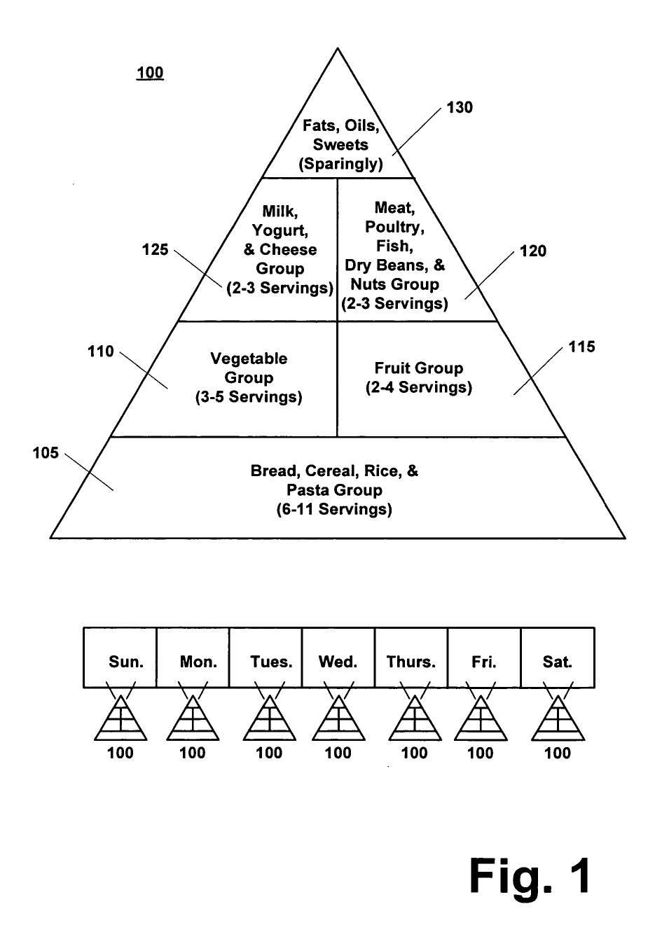

FIG. 1 shows a block diagram of a prior art health management approach. The health management approach is based on the United States Department of Agriculture's “Food Guide Pyramid” 100. Food Guide Pyramid (FGP) 100 provides an outline of what to eat each day. As is shown in FIG. 1, FGP 100 comprises several food type groups 105, 110, 115, 120, 125, and 130, each group providing a recommended daily serving amount. Food type group 105 comprises breads, cereals, and pasta groups having a recommended serving amount of 6-11 servings per day. Vegetable food group 110 recommends 3-5 servings of vegetables per day. Fruit group 115 recommends 2-4 servings of fruits per day. Food group 120 comprises meat, poultry, fish, dry beans and nuts having a recommended serving amount of 2-3 servings per day. Food group 125 comprises milk, yogurt and cheese having a recommended serving amount of 2-3 servings per day. Lastly, FGP 100 comprises food group 130 standing atop of FGP 100 which comprises fats, oils, and sweets. FGP 100 suggests that the foods found in food group 130 be eaten sparingly on a daily basis.

In practice, the foods found in FGP 100 are consumed according to the recommended serving amounts. The exact serving amounts and types of food within each food group varies according to the age, height, and weight of the participating health management subject. Moreover, FGP 100 is put into practice from day to day, week to week, month to month, year to year, or until certain health management goals and milestones have been achieved.

Although FGP 100 is not a rigid prescription, it provides health management principals which, if followed, is purported to help participating subjects to achieve their desired health management goals It is appreciated, however, that although extremely flexible in the amount and type of foods participating health management subjects can intake, that such prior art health management approach may be ineffective in triggering the above-described benefits derived from calorie reduced health management approaches.

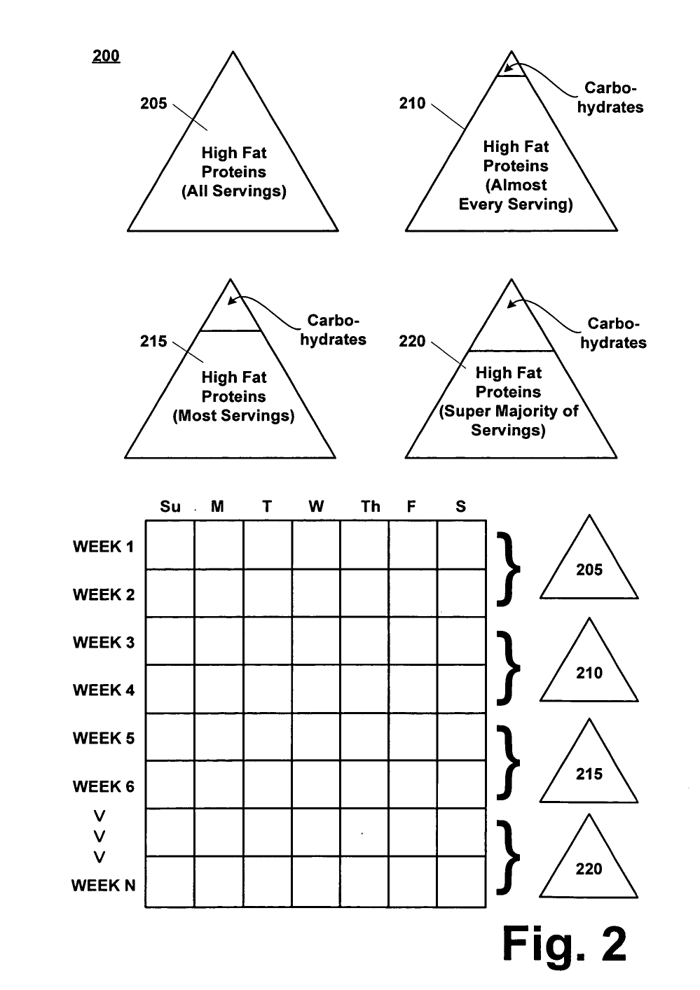

FIG. 2 shows a block diagram of a second prior art health management approach. As is shown, prior art health management approach 200 provides four recommended food intake triangles 205, 210, 215, and 220 that recommend the food types to be consumed for a particular meal or over a given day. The food types in food intake triangles 205, 210, 215, and 200, respectively, generally comprise high fat proteins, and carbohydrates in varying proportions. Specifically, food intake triangle 205 recommends the intake of high fat proteins exclusively. Comparatively, food intake triangle 210 recommends the intake of mostly high fats proteins (almost every serving) with very limited carbohydrate intake. Food intake triangle 215 is similar to food intake triangle 210, however recommending a slightly larger portion of carbohydrate intake (i.e. high fat proteins for most food intake servings). Lastly, food intake 220 triangle shows that carbohydrates account for a growing percentage of the total food intake.

In practice, health management approach 200 recommends that the participating health management subject intake a particular food intake triangle with its prescribed food type percentages for varying periods of time. For example, as is shown in the bottom of FIG. 2, health management approach 200 recommends that food intake triangle 205 be consumed for the first two weeks of the health management approach 200 program. After the first two weeks, participating health management participants are asked to consume the foods in the proportions prescribed by food intake triangle 210 for the next period of time (e.g. week 3 and week 4). For the next time period (e.g. week 5 and week 6), health management approach 200 suggests that participating users consume food in the proportions prescribed by food intake triangle 215. Lastly, the participating user is directed to consume food in the proportions prescribed by food triangle 220 for the remainder of the health management approach 200 program time.

Commercial implementations of these health management approaches include but are not limited to the ATKINS DIET and the SOUTH BEACH DIET. Such health management approaches have been chronicled as effective in assisting participants to lose and/or maintain weight. It is appreciated, however, that health management approach 200 places significant food-type restrictions on participating subjects which does not promote long term compliance and does not trigger the above-described benefits realized from calorie restriction.

Calorie Restricted Health Management Paradigm:

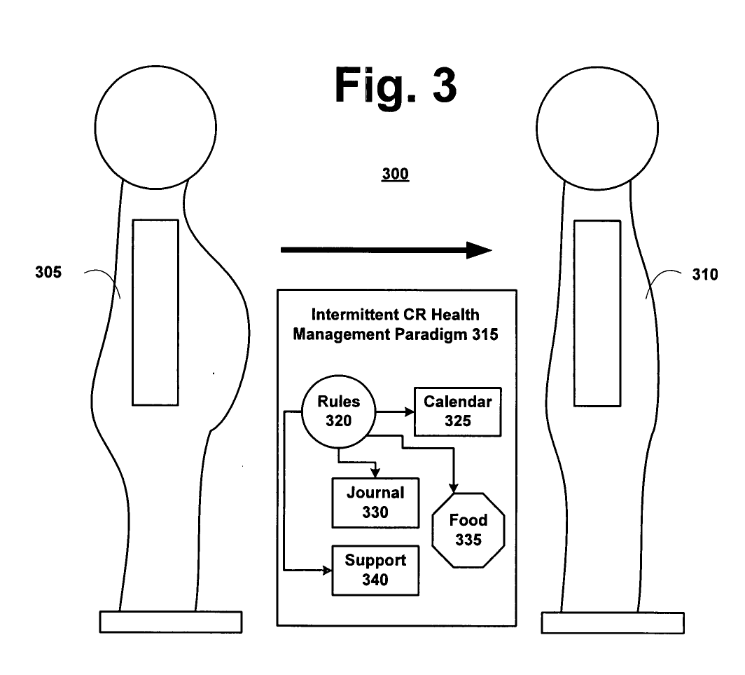

FIG. 3 shows the components of an exemplary calorie restricted (CR) health management environment 300 premised on intermittent calorie restricted feeding. As is shown, CR health management environment 300 comprises a health management participant being in a first health state 305 (e.g., weight state) and the health management participant having a second health state 310 after practicing intermittent CR health management paradigm 315. First health state 305 shows the health management participant as being portly, wherein second weight health 310 shows the health management participant having a more healthy body shape.

Furthermore, as is shown in FIG. 3, intermittent CR health management paradigm 315 comprises rules 320, calendar 325, journal 330, food 335, and support 340. In operation, rules 320 provide the health management participant instructions for using the calendar 325, journal 330, and for the intake of food 335. Additionally, rules 320 provide the health management participant with guidance of how to use and rely on support 340.

In an illustrative implementation, rules 320 may provide the participating health management user with instructions on how to consume food 335 according to selected calendar 325 and when to record the consumption of food 335 in journal 330. In this illustrative implementation, rules 320 can also provide guidance to the health management participant regarding how to employ support so as to adhere to the consumption and time rules. Support 340 may include but is not limited to, food supplements (or replacements), psychological counseling, health counseling, and food consumption protocols.

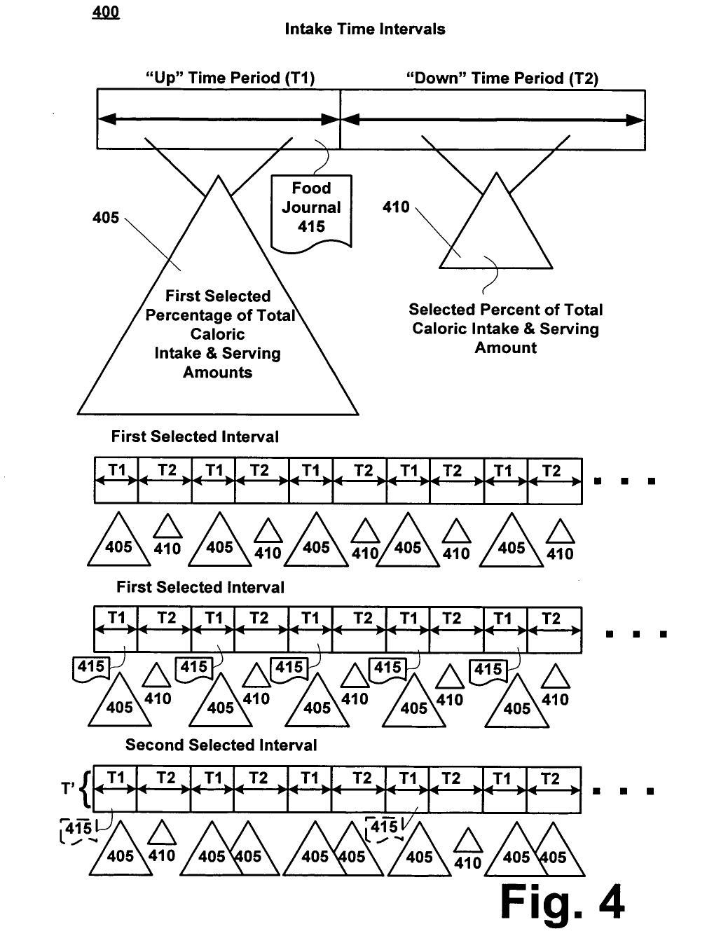

FIG. 4 shows the application of an exemplary intermittent CR health management paradigm 315. As is shown, intermittent calorie restricted health management paradigm application 400 comprises intake time intervals T1 (“Up” time period) and T2 (“Down” timer period). Additionally, intermittent calorie restricted health management paradigm application 400 further comprises food journal 415, first selected percentage of a total caloric intake and serving amounts 405 for consumption during time period T1, and a second selected percent of a total caloric intake and serving amount 410 for consumption during time period T2. Furthermore, as is shown in FIG. 4, intermittent calorie restricted health management application 400 maintains several modes (portions). The portions described include adaptation time period, weight loss time period, and weight maintenance time period.

In operation, if the health management participant (not shown) is engaged in a first selected health corrective interval (i.e., a time period selected to achieve first selected health management goals (not shown)), intermittent calorie restricted health management paradigm application 400 directs the health management participant (not shown) to consume the first selected percentage of the total caloric intake and servings 405 during time period T1 and the second selected percentage of the total caloric intake 410 during time period T2. As is further shown, such pattern is repeated by the health management participant (not shown) until the one or more health management goals and milestones have been reached (as indicated by the ellipses). During the selected heath corrective time period, however, calorie restricted health management paradigm application 400 directs the participant (not shown) to chronicle their food intake in journal 415 during each time period T1 during the duration of the health corrective time period.

Comparatively, during a second selected interval (i.e., a time period selected to achieve second selected health management goals (not shown)), as is shown in FIG. 4, health management paradigm application 400 directs the health management participant (not shown) to consume first selected percentage of the total caloric intake 405 during time period T1 and second selected percentage of the total caloric intake 410 during time period T2, and to repeat such practice for a selected number of times in a given time period T′. Furthermore, as is shown, for those time periods T1 and T2 in the second selected interval T′ when not practicing the intermittent calorie restricted health management paradigm, the participant (not shown) is directed to consume first selected percentage of the total caloric intake 405. As such, the participant (not shown) is consuming the first selected percentage of the total caloric intake more frequently. Additionally, similar to the practice of the first selected interval and as is shown, intermittent calorie restricted health management paradigm application 400 prescribes that the participant (not shown) chronicle the food intake in journal 415.

It is appreciated that although the intermittent calorie restricted health management paradigm can operate such that participating subjects (not shown) can observe weight loss as a collateral benefit of the intermittent CR health management paradigm when the average of the caloric intake over time period T′ is less than 100% (e.g., 100% during T1 and 70% during T2). It is further appreciated that participating subjects can also observe health benefits without weight loss when the average of the caloric intake over time period T′ equals 100% (e.g., 130% during T1 and 70% during T2). It is further appreciated that the health benefits can be realized with or without weight loss such that during a selected period within T′ the participating subject ingests a selected percentage of a total caloric intake having a range of 0-70%.

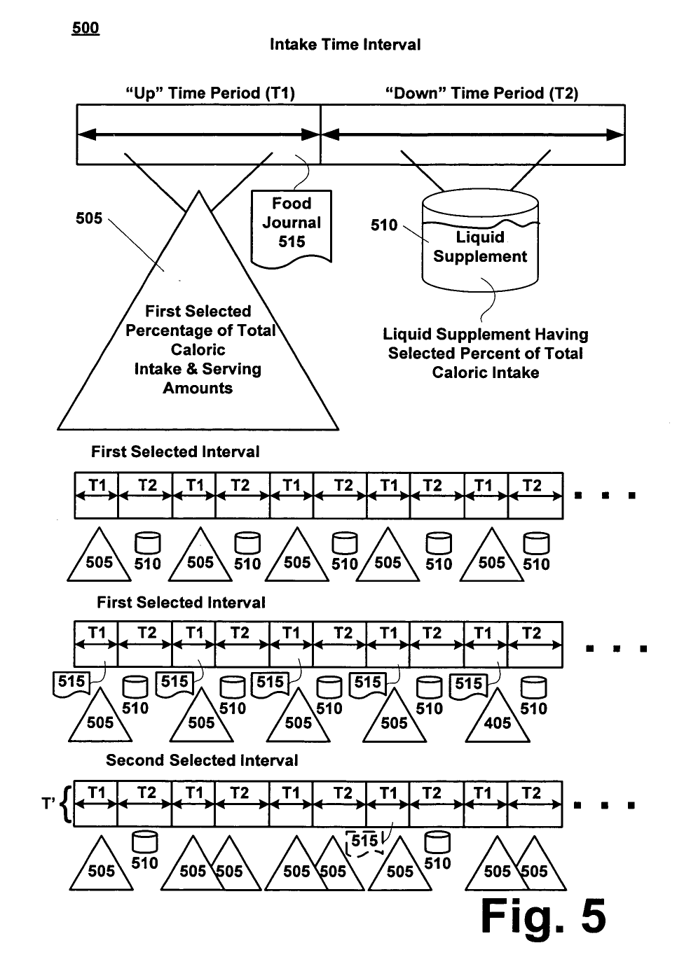

FIG. 5 shows another illustrative application of intermittent CR health management paradigm 315. As is shown, health management paradigm application 500 comprises intake time intervals T1 (“Up” time period) and T2 (“Down” time period). Additionally, health management paradigm application 500 further health management journal 515, first selected percentage of a total caloric intake and serving amounts 505 for consumption during time period T1, and a liquid supplement 510 having a second selected percent of a total caloric intake and serving amount for consumption during time period T2. Furthermore, as is shown in FIG. 5, health management application 500 maintains several modes (portions). The portions described include first selected interval and second selected interval.

In operation, if the health management participant is engaged in the first selected interval, intermittent CR health management paradigm application 500 directs the health management participant (not shown) to consume the first selected percentage of the total caloric intake and servings 505 during time period T1 and the liquid supplement 510 having a second selected percentage of the total caloric intake during time period T2. As is further shown, such pattern is repeated by the participant (not shown) until the first selected interval has expired or until one or more health management goals and milestones have been reached (as indicated by the ellipses). During a portion of the first selected interval, intermittent CR health management paradigm application 500 directs the participant (not shown) to chronicle their food intake in journal 515 during each time period T1 of the portion of the first selected interval.

Comparatively, during the second selected interval, as is shown, health management paradigm application 500 directs the health management participant (not shown) to consume first selected percentage of the total caloric intake 505 during time period T1 and liquid supplement 510 having a second selected percentage of the total caloric intake during time period T2, and to repeat such practice for a selected number of times in second selected interval T′. Furthermore, as is shown, for those time periods T1 and T2 in the weight maintenance in second selected interval T′ when not practicing the intermittent CR health management paradigm, the participant (not shown) is directed to consume first selected percentage of the total caloric intake 505. As such, the participant (not shown) is consuming the first selected percentage of the total caloric intake more frequently. Additionally, similar to the directions provided by the intermittent CR health management paradigm during the first selected interval and, as is shown, intermittent CR health management paradigm application 500 prescribes that the participant (not shown) chronicle the food intake in health management journal 515.

It is appreciated that the value for the total caloric intake as provided in FIGS. 4 and 5 may be obtained from conventional dietary guidelines including published guidelines from the United States Department of Agriculture. These guidelines prescribe the total amount of calories to consume in a given day. In the implementation provided, the inventive concepts described herein contemplate the selection of values for the first and selected percentages of the total caloric to be such that the average of the first and second selected percentages fall within a define percentage range. For example, in the implementation provided, the first selected percentage may be selected to have a value of 100% of a recommend daily caloric intake, wherein the second selected percentage can be a range of 10-70% of a recommended daily total caloric intake. Accordingly, the average percentage of caloric intake over the provided time periods T1 and T2 would fall within the range of 55-85%. Also, the values selected for time periods T1 and T2 are such that T2 is greater than T1. In an illustrative implementation, T1 may be a period of 18 hours, wherein T2 is a period of 30 hours. Lastly, when engaged in selected health management mode (e.g., weight maintenance), participant can be directed to perform a select number of fasts over a selected time period. In an illustrative implementation, to maintain the selected health management mode of weight maintenance, the participant can be directed to perform 2 “down” modified fast days (e.g. 60 hours) in a given week.

It is further appreciated that although the inventive concepts in FIGS. 4 and 5 have been described, by way of example, of having particular values that such values are merely exemplary since the inventive concepts described herein may apply to selected percentages having various values and ranges. Furthermore, it appreciated that although the intermittent restricted calorie health management paradigm is described with an illustrative implementation for health maintenance in the context of weight management that such description is merely illustrative as the inventive concepts described herein can apply to various health management goals.

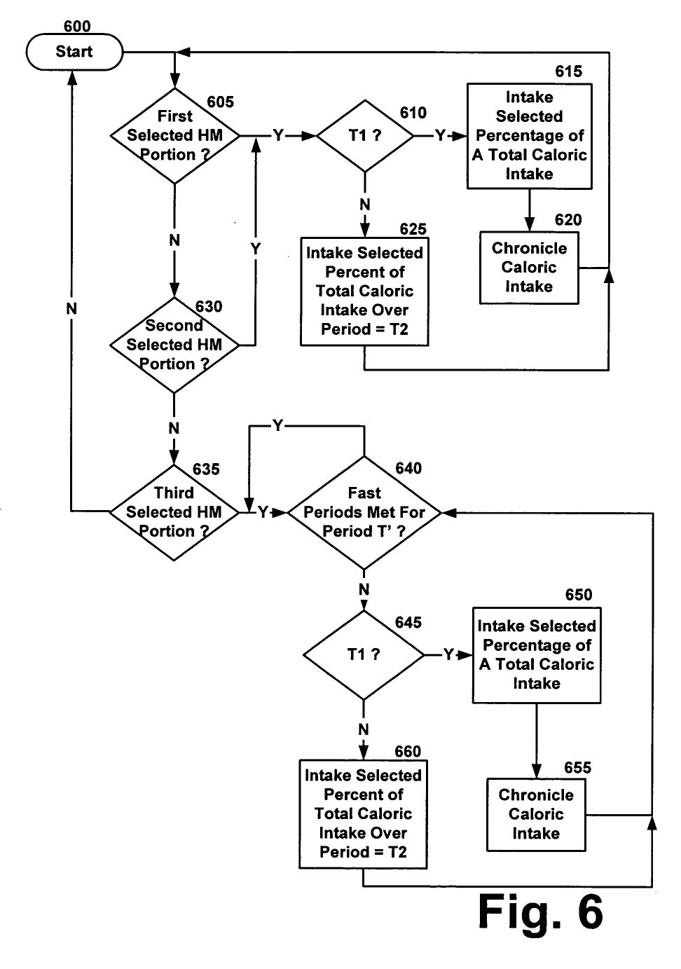

FIG. 6 shows the acts performed when applying intermittent calorie restricted health management paradigm 315. As is shown, health management paradigm 315 begins at block 600 and proceeds to block 605 where a check is performed to determine if the health management participant is engaged in a first selected health management portion (e.g., engaged in efforts to realize first selected health management goals—i.e., adaptation to a weight management protocol) of the intermittent calorie restricted health management paradigm. If the health management participant is engaged in the first selected health management portion of the intermittent calorie restricted health management paradigm, the paradigm proceeds to block 610 where a check is performed to determine if the health management paradigm in within the selected time period T1. If the check at block 610 indicates that it is time period T1, the paradigm proceeds to block 615 where the participant is directed to consume a selected percentage of a total caloric intake. The paradigm then directs the participant to chronicle the caloric intake of block 615 at block 620. From there the health management paradigm processing reverts to block 600 and continues onward.

If, however, the check at block 610 indicates that the paradigm is not within the selected time period T1, it is presumed that the paradigm is within the selected time period T2 (not shown) and the participant is directed to intake a second selected percentage of the total caloric intake over time period T2. From there, the paradigm reverts to block 600 and continues onward.

In the instance, however, it is determined that at block 605, the health management participant is not engaged in the first selected health management portion of the intermittent calorie restricted health management paradigm, the paradigm proceeds to block 630 where a check is performed to determine if the health management participant is engaged in a second selected health management portion (e.g., weight loss portion) of the intermittent calorie restricted health management paradigm. If the check at block 630 indicates that the participant is engaged in the second selected health management portion, the paradigm reverts to block 610 and continues from there.

However, if at block 630, it is determined that health management participant is not engaged in the second selected health management portion of the health management paradigm, the paradigm proceeds to block 635 where a check is performed to determine if the health management participant is engaged in a third selected health management portion (e.g., weight maintenance) of the health management paradigm. If the check at block 635 indicates that the health management participant is not engaged in the third selected health management portion of the health management paradigm, the paradigm reverts to block 600 and continues there from.

In the instance, however, it is determined that the health management participant is not engaged in the third selected health management portion of the intermittent calorie restricted health management paradigm, the paradigm proceeds to block 640 where a check is performed to determine if the health management participant has already performed the recommended fasting periods for a selected period of time T′. If the check at block 640 is true, processing reverts back to the input of block 640 and continues there from. However, if the check at block 640 returns a false result, that is, the health management participant has not performed the recommended “low intake” periods for the selected period of time T′, the intermittent calorie restricted health management paradigm proceeds to block 645 where a check is performed to ascertain if the health management paradigm is within the selected period of time T1. If the check at block 645 indicates that the health management paradigm is within the selected period of time T1, the paradigm proceeds to block 650 where the paradigm directs the health management participant to consume a first selected percentage of a total caloric intake. From there, the paradigm directs the participant to chronicle the caloric intake at block 655. The paradigm then reverts to block 640 and continues from there.

However, if at block 645 it is determined that the paradigm is not within the selected time period T1, it is presumed that the paradigm is within the selected time period T2 (not shown) and the participant is directed to intake a second selected percentage of the total caloric intake over time period T2. From there, the paradigm reverts to block 640 and continues there from.

It is appreciated that although illustrative intermittent calorie restricted health management paradigm is shown to have a first, second, and third selected health management portions that such description is merely illustrative as the inventive concepts described herein can apply to intermittent calorie restricted health management paradigms having various portions for use in achieving a variety of health management goals (e.g., impact disease) other than weight management.

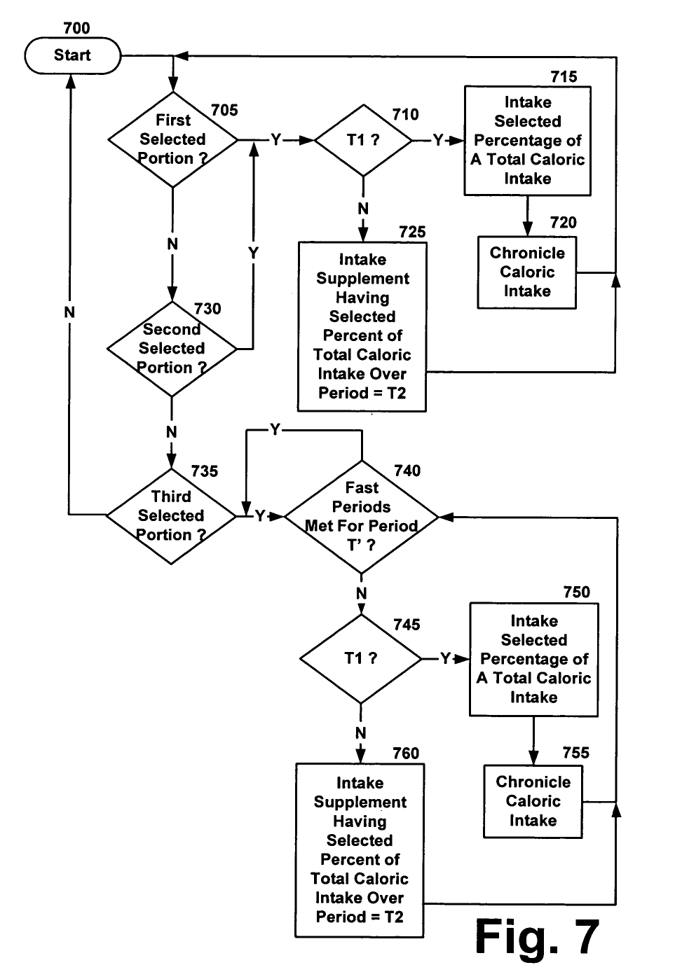

FIG. 7 shows the acts performed in an alternate implementation when applying intermittent calorie restricted health management paradigm 315. As is shown, intermittent calorie restricted health management paradigm 315 begins at block 700 and proceeds to block 705 where a check is performed to determine if the health management participant is engaged in the first selected health management portion (e.g., adaptation) of the intermittent calorie restricted health management paradigm. If the health management participant is engaged in the first selected health management portion of the intermittent calorie restricted health management paradigm, the paradigm proceeds to block 710 where a check is performed to determine if the health management paradigm in within the selected time period T1. If the check at block 710 indicates that it is time period T1, the paradigm proceeds to block 715 where the participant is directed to consume a selected percentage of a total caloric intake. The paradigm then directs the participant to chronicle the caloric intake of block 715 at block 720. From there the health management paradigm processing reverts to block 700 and continues onward.

If, however, the check at block 710 indicates that the paradigm is not within the selected time period T1, it is presumed that the paradigm is within the selected time period T2 (not shown) and the participant is directed to intake a supplement having a second selected percentage of the total caloric intake over time period T2. From there, the paradigm reverts to block 700 and continues onward.

In the instance, however, it is determined that at block 705, the health management participant is not engaged in first selected portion of the intermittent CR health management paradigm (e.g., adaptation portion of a weight management paradigm), the paradigm proceeds to block 730 where a check is performed to determine if the health management participant is engaged in second selected portion of the intermittent CR health management paradigm (e.g., weight loss portion of a weight management paradigm). If the check at block 730 indicates that the participant is engaged in the second selected portion of the paradigm, the paradigm reverts to block 710 and continues from there.

However, if at block 730, it is determined that health management participant is not engaged in the second selected portion of the intermittent CR health management paradigm, the paradigm proceeds to block 735 where a check is performed to determine if the health management participant is engaged in the third selected portion of the intermittent CR health management paradigm. If the check at block 735 indicates that the health management participant is not engaged in the third selected portion of the intermittent CR health management paradigm, the paradigm reverts to block 700 and continues there from.

In the instance, however, it is determined that the health management participant is not engaged in the third selected portion of the intermittent CR health management paradigm, the paradigm proceeds to block 740 where a check is performed to determine if the health management participant has already performed the recommended “low intake” periods for a selected period of time T′. If the check at block 740 is true, processing reverts back to the input of block 740 and continues there from. However, if the check at block 740 returns a false result, that is, the health management participant has not performed the recommended fasting periods for the selected period of time T′, the health management paradigm proceeds to block 745 where a check is performed to ascertain if the health management paradigm is within the selected period of time T1. If the check at block 745 indicates that the health management paradigm is within the selected period of time T1, the paradigm proceeds to block 750 where the paradigm directs the health management participant to consume a first selected percentage of a total caloric intake. From there, the paradigm directs the participant to chronicle the caloric intake at block 755. The paradigm then reverts to block 740 and continues from there.

However, if at block 745 it is determined that the paradigm is not within the selected time period T1, it is presumed that the paradigm is within the selected time period T2 (not shown) and the participant is directed to intake a supplement having a second selected percentage of the total caloric intake over time period T2. From there, the paradigm reverts to block 740 and continues there from.

It is appreciated that although illustrative intermittent calorie restricted health management paradigm is shown to have a first, second, and third selected health management portions that such description is merely illustrative as the inventive concepts described herein can apply to intermittent calorie restricted health management paradigms having various portions for use in achieving a variety of health management goals (e.g., impact disease) other than weight management.

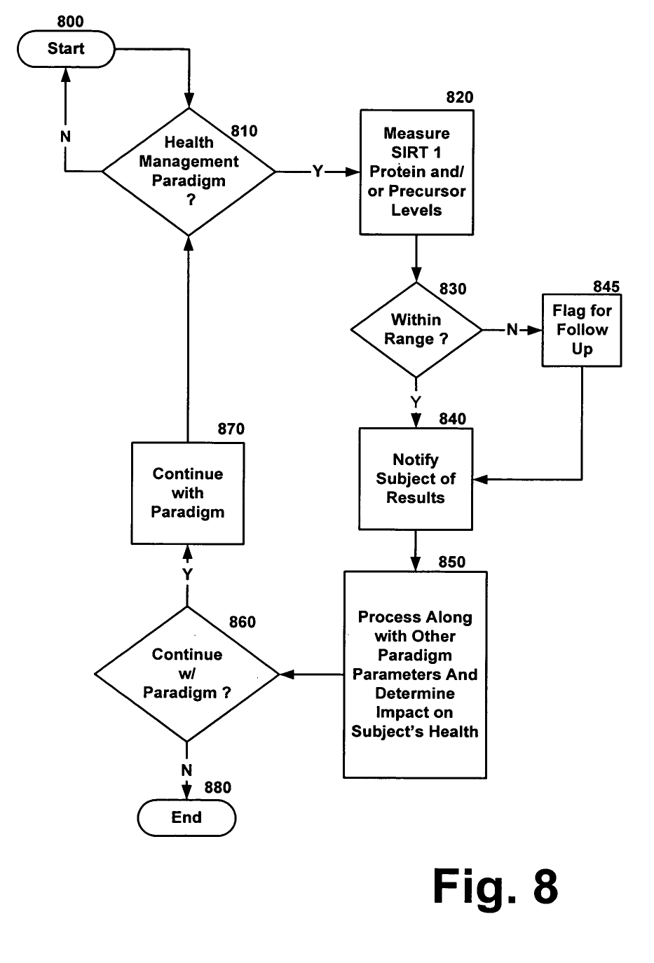

FIG. 8 shows the processing performed when performing monitoring of an exemplary intermittent CR restricted health management paradigm that can be used in health management and/or as part of a disease treatment paradigm for one or more selected diseases and/or health conditions (e.g., leukemia, MS, and Parkinson's disease). As is shown, processing begins at block 800 and then proceeds to block 810 where a check is performed to determine if health monitoring is to be performed. If the check at block 810 indicates that health monitoring is not to occur, processing reverts back to block 800 and proceeds from there.

However, if the check at block 810 indicates that health monitoring is to be performed, processing proceeds to block 820 where the SIRT-1 protein levels and/or a precursor to the production of SIRT-1 are measured. From there, processing proceeds to block 830 where a check is performed to determine whether the SIRT-1 protein and/or precursor are within a selected range. In an illustrative implementation, if a participating subject's SIRT-1 and/or precursor levels show an increase of 40% or more as compared to a baseline test when the participating subject is not being subjected to the health management paradigm, it can be concluded that the health management paradigm is having a beneficial effect on the participating subject. Participating subjects are then informed of the results at block 840. The SIRT-1 and/or precursor measurement is then processed along with other paradigm parameters at block 850. Additionally, the impact on a participating subject's health is determined at block 850.

A check is then performed at block 860 to determine if the participating subject is to continue with the intermittent calorie restricted health management paradigm. If the check at block 860 indicates that the participating subject should not continue with the selected intermittent calorie restricted health management paradigm, processing terminates at block 880. However, if at block 860 it is determined that the participating subject is to continue with the selected intermittent calorie restricted health management paradigm, processing proceeds to block 870 where the intermittent calorie restricted health management paradigm is continued. From there processing reverts to block 810 and proceeds from there.

However, if the check at block 830 indicates that the measured SIRT-1 protein and/or precursor level is not within a selected range, processing proceeds to block 845 where a flag is set to follow up on the protein measurement. From there processing proceeds to block 840 and proceeds from there.

Health Benefits Related to Following an Intermittent Calorie Restricted Health Management Paradigm

Beyond health management in humans, the process described herein is believed to offer other health benefits. As mentioned above, animal studies suggest that CR may contribute to extending the subject's lifespan by triggering one or more genetic markers that are believed to have a direct effect on longevity. The CR program described above is also believed to provide beneficial health effects by modifying the body's reaction to stimuli that cause certain diseases and chronic conditions. These include arthritis (both osteoarthritis and rheumatoid arthritis), bursitis, asthma and bronchitis, allergies, and other inflammatory conditions. Following the CR process of the invention also yields a marked reduction in insulin resistance, which can ameliorate or avoid non-insulin dependant diabetes (NIDDM). The process also can reduce the complications of insulin and non-insulin dependent diabetes, including neuropathy, nephropathy, retinopathy, angiopathy and associated large vessel atherosclerosis.

What is claimed is:

1. A method for accomplishing human weight management comprising:
(a) providing a selected percentage of a total caloric intake during a first time period T1; and
(b) providing a second selected percentage of the total caloric intake during a second time period T2.

2. A dietary process for achieving reduction of the severity and incidence of diseases comprising any of inflammatory conditions and neurological disorders without weight loss comprising:
(a) providing a selected percentage of a total caloric intake during a first time period T1; and
(b) providing a second selected percentage of the total caloric intake during a second time period T2.

3. The method as recited in claim 2 further comprising providing a value for the total caloric intake from one or more dietary guidelines.

4. The method as recited in claim 2 further comprising providing a value for T2 being greater than or equal to a value for T1.

5. The method as recited in claim 2 further comprising repeating steps (a) and (b).

6. The method as recited in claim 2 further comprising providing a percentage of the total caloric intake during time period T1 such that weight loss is not observed.

7. The method as recited in claim 6 further comprising providing a first percentage of the total caloric intake during time period T1 and a second percentage of the total caloric intake during time period T2 such that the average of the first percentage and second percentage is 100%.

8. A method for monitoring health management comprising:
(a) providing a calorie restricted health management paradigm; and
(b) providing a means for monitoring the activity and/or amount of SIRT-1 protein as an indicator of adherence to the calorie restricted health management paradigm.

9. The method as recited in claim 8 further comprising providing a biochemical means for measuring the activity and/or amount of SIRT-1 protein in a participating subject.

10. The method as recited in claim 9 further comprising providing a biochemical test of white blood cells to measure the activity and/or amount of SIRT-1 protein.

11. The method as recited in claim 8 further comprising adjusting the calorie restricted health management paradigm based on the activity and/or amount of SIRT-1 protein that is measured.

12. The method as recited in claim 8 further comprising measuring the activity and/or amount of SIRT-1 protein as a measure of the state of health for a participating subject.

13. A method for treating diseases using protein monitoring in mammals comprising:
(a) providing an intermittent calorie restricted health management paradigm; and
(b) providing a means for monitoring the activity and/or amount of SIRT-1 as an indicator of the acuity of one or more diseases in a participating subject.

14. The method as recited in claim 13 further comprising providing a biochemical means for measuring the activity and/or amount of SIRT-1 protein in the participating subject.

15. The method as recited in claim 14 further comprising providing a biochemical test of white blood cells to measure the activity and/or amount of SIRT-1 protein.

16. The method as recited in claim 13 further comprising adjusting the treatment of one or more diseases based on the activity and/or amount of SIRT-1 protein that is measured.

17. The method as recited in claim 13 further comprising measuring the activity and/or amount of SIRT-1 protein as a measure of the state of health for a participating subject.

18. The method as recited in claim 14 further comprising monitoring the SIRT-1 protein to create a base line measurement for a participating subject.

19. The method as recited in claim 18 further comprising monitoring the SIRT-1 protein such that an increase in the range of 20% to 50% of the SIRT-1 protein as compared with the base line measurement triggers one more health management decisions.

20. The method as recited in claim 19 further comprising providing a health management decision to a participating subject comprising any of stopping the intermittent calorie restricted health management paradigm, adjusting the intermittent calorie restricted health management paradigm, and maintaining the intermittent calorie restricted health management paradigm.

21. The method as recited in claim 20 further comprising providing an intermittent calorie restricted health management paradigm comprising intaking within a range between 10% to 70% of a total caloric intake for a first portion of a selected time period and intaking within a range between 10%-100% of total caloric intake for a second portion of the selected time period.

22. A method for treating diseases using gene monitoring in mammals comprising:
(a) providing an intermittent calorie restricted health management paradigm; and
(b) providing a means for monitoring the activity and/or amount of PNC-1 as an indicator of the acuity of one or more diseases in a participating subject.

23. The method as recited in claim 22 further comprising providing a biochemical means for measuring the activity and/or amount of PNC-1 gene in the participating subject.

24. The method as recited in claim 23 further comprising providing a biochemical test of white blood cells to measure the activity and/or amount of the PNC-1 gene.

25. The method as recited in claim 12 further comprising adjusting the treatment of one or more diseases based on the presence of the PNC-1 gene.

25. The method as recited in claim 22 further comprising measuring the presence of the PNC-1 gene as a measure of the state of health for a participating subject.

26. The method as recited in claim 23 further comprising monitoring the presence of the PNC-1 gene to create a base line measurement for a participating subject.

27. The method as recited in claim 26 further comprising monitoring the presence of the PNC-1 gene as compared with the base line measurement triggers one more health management decisions.

28. The method as recited in claim 27 further comprising providing a health management decision to a participating subject comprising any of stopping the intermittent calorie restricted health management paradigm, adjusting the intermittent calorie restricted health management paradigm, and maintaining the intermittent calorie restricted health management paradigm.

29. The method as recited in claim 28 further comprising providing an intermittent calorie restricted health management paradigm comprising intaking within a range between 10% to 70% of a total caloric intake for a first portion of a selected time period and intaking within a range between 10%-100% of total caloric intake for a second portion of the selected time period.
